(19) United States
(12) Patent Application Publication
(10) Pub. No.: US 2026/0069469 A1
(43) Pub. Date: Mar. 12, 2026

(54) GUSSET PERFORMANCE STRUCTURE AND GARMENT INCLUDING SAME

(71) Applicant: Thinx Inc., Neenah, WI (US)

(72) Inventors: Courtney Newman, Beacon, NY (US); Oxana Razdolski, Franklin Lakes, NJ (US); Joyce Baran, Stratord, CT (US)

(73) Assignee: Thinx Inc., Neenah, WI (US)

(21) Appl. No.: 19/106,157

(22) PCT Filed: Aug. 25, 2023
(86) PCT No.: PCT/US2023/031163

§ 371 Date: Feb. 24, 2025

Related U.S. Application Data
(60) Provisional application No. 63/401,249, filed on Aug. 26, 2022.

(51) Int. Cl. A61F 13/494; A41B 9/00

(52) U.S. Cl. CPC A61F 13/4942; A41B 9/004; A41B 2400/62

(57) ABSTRACT

An article including: a first part; and a second part. The first part includes: at least one top layer having a first edge portion: at least one intermediate layer having a second edge portion; and at least one back layer having a third edge portion. The second part includes: a first curved portion: an extension portion; and a second curved portion. The extension portion extends from the first curved portion and from the second curved portion, and the first curved portion overlaps a top surface of at least the first edge portion of the at least one top layer and the second curved portion overlaps a bottom surface of at least the third edge portion of the at least one back layer.

### CROSS REFERENCE

This patent application claims the benefit of U.S. Provisional Patent Application 63/401,249 filed 26 Aug. 2022, which is incorporated by reference herein for all purposes.

### TECHNICAL FIELD

This disclosure relates to a gusset performance structure that includes both surface wicking and a one-way moisture transport to an inner layer to improve moisture absorbency and comfort for a user, and that is capable of being structured to be held securely in place without any visible seamlines, and a garment including the gusset performance structure.

### BACKGROUND

In this disclosure, where a document, an act, and/or an item of knowledge is referred to and/or discussed, then such reference and/or discussion is not an admission that the document, the act, and/or the item of knowledge and/or any combination thereof was at a priority date, publicly available, known to a public, part of common general knowledge, and/or otherwise constitutes any prior art under any applicable statutory provisions, and/or is known to be relevant to any attempt to solve any problem with which this disclosure is concerned with. Further, nothing is disclaimed.

There is a desire to enable a gusset performance structure that includes both surface wicking and a one-way moisture transport to an inner layer to improve moisture absorbency and comfort for a user, and that is capable of being structured to be held securely in place without any visible seamlines. However, despite various attempts by others, this type of absorptive or incontinence and menstrual underwear or garment does not yet exist.

### SUMMARY

This disclosure enables an absorptive or incontinence or menstrual underwear and/or garment that is sufficiently waterproof, and capable of being structured to be held securely in place without any visible seamlines. This disclosure at least partially addresses at least one of above inefficiencies. However, this disclosure can prove useful to other technical areas. Therefore, various claims recited below should not be construed as necessarily limited to addressing any of the above inefficiencies.

An exemplary embodiment of this disclosure is directed to an article comprising: a first part; and a second part, wherein the first part comprises: at least one top layer; at least one intermediate layer; and at least one back layer, and wherein the second part comprises: a curved portion; and an extension portion, wherein the extension portion extends from the curved portion, and wherein a top part of the curved portion overlaps a top surface of at least the edge portion of the at least one top layer and a bottom part of the curved portion overlaps a bottom surface of an at least an edge portion of the at least one back layer.

In an exemplary embodiment, the edge portion of each of the at least one top layer and the at least one back layer and an edge portion of the at least one intermediate layer are connected via a first bonding component.

In an exemplary embodiment, the top portion of the second part is connected to the at least one top layer via a second bonding component.

In an exemplary embodiment, the bottom portion of the second part is connected to at least one back layer via a third bonding component.

In an exemplary embodiment, the extension part of the second part is folded in a downward direction such that the extension part curves under an outermost bottom surface of the first part such that a top surface of the extension part forms a secondary bottom surface of the extension part.

In an exemplary embodiment, the extension part comprises two layers that are connected via a fourth bonding component. The two layers of the extension part can be disjointed or connected and can be connected to the curved part in one or many positions.

In an exemplary embodiment, the first to fourth bond components are the same or different, and are independently selected from bonding tape, adhesive, glue or combinations thereof.

In an exemplary embodiment, the article further comprises a material layer, wherein an edge portion of the material layer is folded in an upward direction to form a second folded portion such that a bottom surface of the material layer forms a secondary top surface of the material layer, and wherein the secondary top surface of the material layer is connected to the secondary bottom surface of the extension part via a stitching pattern.

In another exemplary embodiment, the at least one top layer is a wicking layer. A material of the wicking layer is selected from fabric, natural yarn, synthetic yarn, synthetic fabric, polyurethane, wool, polypropylene, polyamide, polyester or combinations thereof.

In another exemplary embodiment, the at least one back layer is a waterproof layer. A material of the waterproof layer is selected from a water resistant, a water repellant material or a combination thereof.

In another exemplary embodiment, a material of the at least one intermediate layer is selected from fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-static®, Tencel™, lyocell, modal, or combinations thereof.

In another exemplary embodiment, a material of the material layer is selected from fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-static®, Tencel™, lyocell, modal, or combinations thereof.

In another exemplary embodiment, a weight of the article is about 50 g to about 500 g.

In another exemplary embodiment, a width of the article is about 0.5 inches to about 6.0 inches at its narrowest point and about 3.0 inches to about 14.0 inches at its widest point.

In another exemplary embodiment, a water absorption capacity of the article is about 250 ml to about 1000 ml.

Another exemplary embodiment of this disclosure is directed to a garment comprising the article described herein.

Another exemplary embodiment of this disclosure is directed to a device comprising a garment. The garment includes a crotch area including the article described in any of the prior exemplary embodiments, a front body portion, a rear body portion, and a pair of leg openings.

Another exemplary embodiment is directed to a device that further comprises an elastic waistband.

Another exemplary embodiment is directed to a device that further comprises a label.

In another exemplary embodiment, each of the pair of leg openings of any of the prior exemplary embodiments is surrounded by an elastic layer folded into the body portion and connected to the body portion via a leg opening bonding component.

In another exemplary embodiment, the front body portion is connected to the crotch area via a front bonding component, and/or the front body portion is connected to the crotch area via a rear bonding component.

In another exemplary embodiment, the front bonding component, the rear bonding component and the leg opening component are the same or different and can be selecting from a stitching, a knitting, an adhesive layer, a bonding tape, or combinations thereof.

Another exemplary embodiment of this disclosure is directed to a method comprising: causing a garment to include a crotch area; causing the crotch area to comprise: a first part; and a second part; causing the first part to comprise: at least one top layer; at least one intermediate layer; and at least one back layer; causing the second part to comprise: a curved portion, and an extension portion; causing the extension portion to extend from the curved portion; and causing a top part of the curved portion to overlap a top surface of at least the edge portion of the at least one top layer and a bottom part of the curved portion to overlap a bottom surface of an at least an edge portion of the at least one back layer.

It should be understood that the various individual aspects and features of this disclosure can be combined with any one or more individual aspect or feature, in any number, to form embodiments of the present invention that are specifically contemplated and encompassed by the present invention.

This disclosure is embodied in various forms illustrated in a set of accompanying illustrative drawings. Variations are contemplated as being a part of this disclosure, limited only by scope of various claims recited herein.

### DESCRIPTION OF DRAWINGS

The set of accompanying illustrative drawings shows various exemplary embodiments of this disclosure. Such drawings are not to be construed as necessarily limiting this disclosure. Like numbers and/or similar numbering scheme can refer to like and/or similar elements throughout.

FIG. 1 is a front view of a gusset performance structure according to an exemplary embodiment of the subject application.

FIG. 2A-FIG. 2D are illustrations of a garment including a gusset performance structure according to an exemplary embodiment of the subject application.

FIG. 3 is a front view of a gusset performance structure according to an exemplary embodiment of the subject application.

### DETAILED DESCRIPTION OF PREFERRED EMBODIMENTS

This disclosure is now described more fully with reference to the set of accompanying illustrative drawings, in which exemplary embodiments of this disclosure are shown. This disclosure can be embodied in many different forms and should not be construed as necessarily being limited to the exemplary embodiments disclosed herein. Rather, the exemplary embodiments are provided so that this disclosure is thorough and complete, and fully conveys various concepts of this disclosure to those skilled in a relevant art.

Features described with respect to certain exemplary embodiments can be combined and sub-combined in and/or with features described with respect to various other exemplary embodiments, even if such combinations and/or sub-combinations are not specifically described herein. Also, different aspects and/or elements of exemplary embodiments, as disclosed herein, can be combined and sub-combined in a similar manner as well. Further, some exemplary embodiments, whether individually and/or collectively, can be components of a larger system, wherein other procedures can take precedence over and/or otherwise modify their application. Additionally, a number of steps can be required before, after, and/or concurrently with exemplary embodiments, as disclosed herein. Note that any and/or all methods and/or processes, at least as disclosed herein, can be at least partially performed via at least one entity in any manner.

Various terminology used herein can imply direct or indirect, full or partial, temporary or permanent, action or inaction. For example, when an element is referred to as being “on,” “connected” or “coupled” to another element, then the element can be directly on, connected or coupled to the other element and/or intervening elements can be present, including indirect and/or direct variants. In contrast, when an element is referred to as being “directly connected” or “directly coupled” to another element, there are no intervening elements present.

Although the terms first, second, etc, can be used herein to describe various elements, components, regions, layers and/or sections, these elements, components, regions, layers and/or sections should not necessarily be limited by such terms. These terms are used to distinguish one element, component, region, layer or section from another element, component, region, layer or section. Thus, a first element, component, region, layer, or section discussed below could be termed a second element, component, region, layer, or section without departing from various teachings of this disclosure.

Various terminology used herein is for describing particular exemplary embodiments and is not intended to be necessarily limiting of this disclosure. As used herein, various singular forms “a,” “an” and “the” are intended to include various plural forms as well, unless a context clearly indicates otherwise. Various terms “comprises,” “includes,” “comprising” and/or “including” when used in this specification specify a presence of stated features, integers, steps, operations, elements, and/or components, but do not preclude the presence and/or addition of one or more other features, integers, steps, operations, elements, components, and/or groups thereof.

As used herein, a term “or” is intended to mean an inclusive “or” rather than an exclusive “or.” That is, unless specified otherwise, or clear from context, “X employs A or B” is intended to mean any of a set of natural inclusive permutations. That is, if X employs A; X employs B; or X employs both A and B, then “X employs A or B” is satisfied under any of the foregoing instances.

Exemplary embodiments of this disclosure are described herein with reference to illustrations of idealized embodiments (and intermediate structures) of this disclosure. As such, variations from various illustrated shapes as a result, for example, of manufacturing techniques and/or tolerances, are to be expected. Thus, various exemplary embodiments of this disclosure should not be construed as necessarily limited to various particular shapes of regions illustrated herein, but are to include deviations in shapes that result, for example, from manufacturing.

Any and/or all elements, as disclosed herein, can be formed from a same, structurally continuous piece, such as being unitary, and/or be separately manufactured and/or connected, such as being an assembly and/or modules. Any and/or all elements, as disclosed herein, can be manufactured via any manufacturing processes, whether additive manufacturing, subtractive manufacturing, and/or other any other types of manufacturing. For example, some manufacturing processes include three dimensional (3D) printing, laser cutting, computer numerical control routing, milling, pressing, stamping, vacuum forming, hydroforming, injection molding, lithography, and so forth.

Any and/or all elements, as disclosed herein, can be and/or include, whether partially and/or fully, a solid, including a metal, a mineral, an amorphous material, a ceramic, a glass ceramic, an organic solid, such as wood and/or a polymer, such as rubber, a composite material, a semiconductor, a nanomaterial, a biomaterial and/or any combinations thereof. Any and/or all elements, as disclosed herein, can be and/or include, whether partially and/or fully, a coating, including an informational coating, such as ink, an adhesive coating, a melt-adhesive coating, such as vacuum seal and/or heat seal, a release coating, such as tape liner, a low surface energy coating, an optical coating, such as for tint, color, hue, saturation, tone, shade, transparency, translucency, opaqueness, luminescence, reflection, phosphorescence, anti-reflection and/or holography, a photo-sensitive coating, an electronic and/or thermal property coating, such as for passivity, insulation, resistance or conduction, a magnetic coating, a water-resistant and/or waterproof coating, a scent coating and/or any combinations thereof. Any and/or all elements, as disclosed herein, can be rigid, flexible, and/or any other combinations thereof. Any and/or all elements, as disclosed herein, can be identical and/or different from each other in material, shape, size, color and/or any measurable dimension, such as length, width, height, depth, area, orientation, perimeter, volume, breadth, density, temperature, resistance, and so forth. Any and/or all elements, as disclosed herein, can be and/or include, any type of fabric, without any limitation as long as they have the required properties as described herein, including but not limited to, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-static®, Tencel™, lyocell, modal, or combinations thereof.

Unless otherwise defined, all terms (including technical and scientific terms) used herein have the same meaning as commonly understood by one of ordinary skill in an art to which this disclosure belongs. Various terms, such as those defined in commonly used dictionaries, should be interpreted as having a meaning that is consistent with a meaning in a context of a relevant art and should not be interpreted in an idealized and/or overly formal sense unless expressly so defined herein.

Furthermore, relative terms such as “below,” “lower,” “above,” and “upper” can be used herein to describe one element's relationship to another element as illustrated in the set of accompanying illustrative drawings. Such relative terms are intended to encompass different orientations of illustrated technologies in addition to an orientation depicted in the set of accompanying illustrative drawings. For example, if a device in the set of accompanying illustrative drawings were turned over, then various elements described as being on a “lower” side of other elements would then be oriented on “upper” sides of other elements. Similarly, if a device in one of illustrative figures were turned over, then various elements described as “below” or “beneath” other elements would then be oriented “above” other elements. Therefore, various example terms “below” and “lower” can encompass both an orientation of above and below.

As used herein, a term “about” and/or “substantially” refers to a +/−10% variation from a nominal value/term. Such variation is always included in any given value/term provided herein, whether or not such variation is specifically referred thereto.

If any disclosures are incorporated herein by reference and such disclosures conflict in part and/or in whole with this disclosure, then to an extent of a conflict, if any, and/or a broader disclosure, and/or broader definition of terms, this disclosure controls. If such disclosures conflict in part and/or in whole with one another, then to an extent of a conflict, if any, a later-dated disclosure controls.

The term “garment” includes without limitation any item that is worn. A garment also includes, without limitation, any item that has a crotch area. A garment includes, for example, an underwear, a brief, an undergarment, a bathing suit, a pajama, a shorts, a leotard, a legging, a pants, a skort, a panty, a boy short, a bikini, a thong, an overalls, a bodysuit, a leotard, a tights, a pantyhose, a boxers, a biking shorts, or a compression shorts. A material of any such garment can include, but is not limited to, fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-static®, Tencel™, lyocell, modal, or combinations thereof.

As used herein, the term “stitching” includes any stitching pattern that can connect two or more layers, without being limited thereto. Any such stitching pattern may be seamless, but is not limited thereto.

As used herein, the term “knitting” includes any knitting pattern that can connect two or more layers, without being limited thereto. Any such knitting pattern may be seamless, but is not limited thereto.

As used here, the term “gusset performance structure” includes a panel, that is inserted into a garment to help shape and reinforce key points, add breadth, reduce stress, and the like, in clothing. A gusset performance structure can be included at any position in a garment, including but not limited to the shoulders, underarms, hemlines, crotches, and the like. The shape of the gusset performance structure is not particularly limited, and can include any suitable shape, including but not limited to a triangular, rhomboidal, diamond shape, and/or as described herein.

Exemplary embodiments of this disclosure are described herein with reference to illustrations of idealized embodiments (and intermediate structures) of this disclosure. As such, this disclosure includes variations from various illustrated shapes as a result, for example, of manufacturing techniques and/or tolerances. Thus, various exemplary embodiments of this disclosure should not be construed as necessarily limited to various particular shapes of regions illustrated herein, but are to include deviations in shapes that result, for example, from manufacturing.

Unless otherwise defined, all terms (including technical and scientific terms) used herein have the same meaning as commonly understood by one of ordinary skill in an art to which this disclosure belongs. Various terms, such as those defined in commonly used dictionaries, should be interpreted as having a meaning that is consistent with a meaning in a context of relevant art and should not be interpreted in an idealized and/or overly formal sense unless expressly so defined herein.

An exemplary embodiment of this disclosure is directed to an article comprising: a first part; and a second part, wherein the first part comprises: at least one top layer; at least one intermediate layer; and at least one back layer, and wherein the second part comprises: a curved portion; and an extension portion, wherein the extension portion extends from the curved portion, and wherein a top part of the curved portion overlaps a top surface of at least the edge portion of the at least one top layer and a bottom part of the curved portion overlaps a bottom surface of an at least an edge portion of the at least one back layer.

In an exemplary embodiment, the edge portion of each of the at least one top layer and the at least one back layer and an edge portion of the at least one intermediate layer are connected via a first bonding component.

In an exemplary embodiment, the top portion of the second part is connected to the at least one top layer via a second bonding component.

In an exemplary embodiment, the bottom portion of the second part is connected to at least one back layer via a third bonding component.

In an exemplary embodiment, the extension part of the second part is folded in a downward direction such that the extension part curves under an outermost bottom surface of the first part such that a top surface of the extension part forms a secondary bottom surface of the extension part.

In an exemplary embodiment, the extension part comprises two layers that are connected via a fourth bonding component. The two layers of the extension part can be disjointed or connected and can be connected to the curved part in one or many positions.

In an exemplary embodiment, the first to fourth bond components are the same or different, and are independently selected from bonding tape, adhesive, glue or combinations thereof.

In an exemplary embodiment, the article further comprises a material layer, wherein an edge portion of the material layer is folded in an upward direction to form a second folded portion such that a bottom surface of the material layer forms a secondary top surface of the material layer, and wherein the secondary top surface of the material layer is connected to the secondary bottom surface of the extension part via a stitching pattern.

In another exemplary embodiment, the at least one top layer is a wicking layer. A material of the wicking layer is selected from fabric, natural yarn, synthetic yarn, synthetic fabric, polyurethane, wool, polypropylene, polyamide, polyester or combinations thereof.

In another exemplary embodiment, the at least one back layer is a waterproof layer. A material of the waterproof layer is selected from a water resistant, a water repellant material or a combination thereof.

In another exemplary embodiment, a material of the at least one intermediate layer is selected from fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-static®, Tencel™, lyocell, modal, or combinations thereof.

In another exemplary embodiment, a material of the material layer is selected from fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-static®, Tencel™, lyocell, modal, or combinations thereof.

In another exemplary embodiment, a weight of the article is about 50 g to about 500 g.

In another exemplary embodiment, a width of the article is about 0.5 inches to about 6.0 inches at its narrowest point and about 3.0 inches to about 14.0 inches at its widest point.

In another exemplary embodiment, a water absorption capacity of the article is about 250 ml to about 1000 ml.

Another exemplary embodiment of this disclosure is directed to a garment comprising the article described herein.

Another exemplary embodiment of this disclosure is directed to a device comprising a garment. The garment includes a crotch area including the article described in any of the prior exemplary embodiments, a front body portion, a rear body portion, and a pair of leg openings.

Another exemplary embodiment is directed to a device that further comprises an elastic waistband.

Another exemplary embodiment is directed to a device that further comprises a label.

In another exemplary embodiment, each of the pair of leg openings of any of the prior exemplary embodiments is surrounded by an elastic layer folded into the body portion and connected to the body portion via a leg opening bonding component.

In another exemplary embodiment, the front body portion is connected to the crotch area via a front bonding component, and/or the front body portion is connected to the crotch area via a rear bonding component.

In another exemplary embodiment, the front bonding component, the rear bonding component and the leg opening component are the same or different and can be selecting from a stitching, a knitting, an adhesive layer, a bonding tape, or combinations thereof.

Another exemplary embodiment of this disclosure is directed to a method comprising: causing a garment to include a crotch area; causing the crotch area to comprise: a first part; and a second part; causing the first part to comprise: at least one top layer; at least one intermediate layer; and at least one back layer; causing the second part to comprise: a curved portion, and an extension portion; causing the extension portion to extend from the curved portion; and causing a top part of the curved portion to overlap a top surface of at least the edge portion of the at least one top layer and a bottom part of the curved portion to overlap a bottom surface of an at least an edge portion of the at least one back layer.

Hereinafter, preferred embodiments of the present disclosure will be described in detail with reference to the accompanying drawings. The configurations disclosed in the preferred embodiments and drawings of the present specification are examples of preferred embodiments of the present disclosure, and thus it should be understood that there can be alternative equivalents or modification examples that can replace the preferred embodiments of this disclosure.

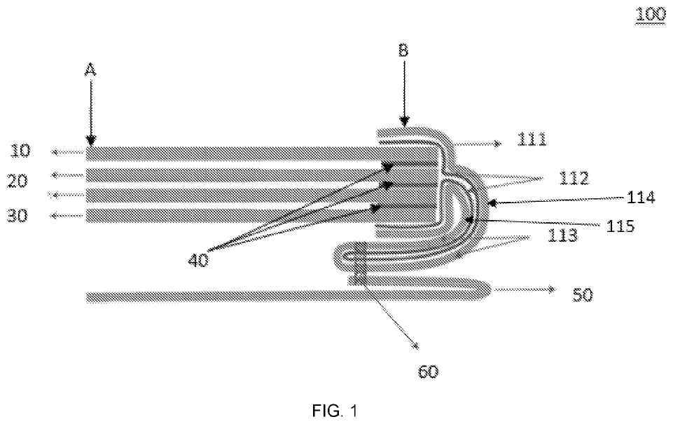

FIG. 1 is a front view of a gusset performance structure according to an exemplary embodiment of the subject application.

As illustrated in FIG. 1, a gusset performance structure 100 includes at least one top layer 10, which faces a user, one or more intermediate layers 20 and at least one back layer 30, which faces a garment. Positions A and B in FIG. 1 correspond to the center of the gusset performance structure and a leg opening of the gusset performance structure of a garment including the gusset performance structure, respectively. An edge portion of each of the at least one top layer 10, one or more intermediate layers 20 and at least one back layer 30 are arranged to be bonded together, via bonding strips 40, at or adjacent to position B of FIG. 1. The top layer 10 includes a suitable moisture-wicking material, including but not limited to, fabric, natural yarn, synthetic yarn, synthetic fabric, polyurethane, wool, polypropylene, polyamide, polyester, or combinations thereof. The moisture-wicking material may be any material that allows one-way transport of moisture, water and/or liquids. A layer comprising the moisture-wicking material may be a water/moisture absorbing layer, such as a polymer blend, including but not limited to a polyester/nylon blend (e.g., 80% polyester, 20% nylon). In various exemplary embodiments, the moisture absorbing layer may include a combination of materials such as, but not limited to, 100% polyester, 96% cotton/4% spandex, 80% polyester/20% nylon, Bramante®/cotton blend, cotton flock, natural fibers, wool, and the like. In various exemplary embodiments, the moisture-wicking material may be a combination of materials selected from fabric, natural yarn, synthetic yarn, synthetic fabric, polyurethane, wool, polypropylene, polyamide, polyester or combinations thereof. The back layer 30 includes a suitable waterproof material, which can be a coating, but a water resistant or water repellant material can be included as an alternate to, or together with, the waterproof material. The intermediate layers 20 include an absorbent material, including but not limited to fabric, natural yarn, synthetic yarn, synthetic fabric, polyurethane, wool, polypropylene, polyamide, polyester, or combinations thereof. Any of layers 10 to 30 can include any suitable material, including but not limited to fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-static®, Tencel™, lyocell, modal, or combinations thereof.

After the edge portion of the top layer 10, the intermediate layers 20 and the back layer 30 are bonded together, a curved portion 111 extends over the edge portions and is bonded to the edge portions via bonding strip 112. A top part of the curved portion 111 overlaps a top surface of at least the edge portion of the at least one top layer 10 and a bottom part of the curved portion 111 overlaps a bottom surface of at least the edge portion of the at least one back layer 30. The curved portion 111 includes an extension portion 113. The extension portion 113 extends from the curved portion, and the extension portion 113 folds under the bottom part of the curved portion 111 that overlaps a bottom surface of at least the edge portion of the at least one back layer 30. The extension portion 113 comprises a first layer 114 and a second layer 115, which run substantially parallel. In certain embodiments, the curved portion 111 and extension portion 113 are formed from a single piece. In other embodiments, the curved portion 111 and the extension portion 113 are formed from two or more pieces. The bonding strip 112 extends between the first layer 114 and the second layer 115 and bonds the first layer 114 to the second layer 115. The bonding strip 112 may comprise a single piece of bonding material that extends from the top part of the curved portion 111 between the first layer 114 and the second layer 115 and continues on to the bottom part of the curved portion 111. In certain embodiments, the bonding strip 112 may comprise multiple portions of bonding material, with one or more portions extending under the top part of the curved portion 111, one or more portions extending under the bottom part of the curved portion 111, and/or one or more portions extending between the first layer 114 and the second layer 115. The first layer 114 and the second layer 115 are folded under the bottom surface of at least the edge portion of the at least one back layer 30, such that the second layer 115 is adjacent the lower part of the curved portion 111 and the at least one back layer 30.

As further illustrated in FIG. 1, each of the top surfaces of layers 20 and 30, faces the bottom surface of adjacent layer. The various layers of the gusset performance structure can be connected to an adjacent layer as illustrated in FIG. 1. For example, as shown in FIG. 1: the top surface of bottom layer 30 can be connected to the back surface of the adjacent intermediate layer 20; two adjacent intermediate layers 20 can be connected; and/or a top surface of the innermost intermediate layer 20, which is closest to the top layer 10, can be connected to the bottom surface of the top layer 10, via a bonding component 40. The bonding component 40 between the various layers can be the same construction or material or different construction or material.

The gusset performance structure shown in FIG. 1 also includes a layer 50, which is connected to the extension portion 113 via bonding element 60. Bonding element 60 can be selected from a stitching, a knitting, an adhesive layer, a bonding tape, or combinations thereof. The layer 50 can include any suitable material for use in a garment, including but not limited to fabric, natural yarn, synthetic yarn, synthetic fabric, polyurethane, wool, polypropylene, polyamide, polyester, or combinations thereof. The bonding element 60 can be placed at any position along the extension portion 113, such as, for example, at or near the tip of the extension portion 113 or at or near the base of the extension portion 113, or at any point in between. In certain embodiments, a plurality of bonding elements 60 may be used. Each of the bonding elements 60 may be placed at different locations, such as, for example, a first bonding element 60 may be placed at or near the tip of the extension portion 113 and a second bonding element 60 may be placed at or near the base of the extension portion 113, or one or more of the first bonding element 60 or the second bonding element 60 may be placed in between those locations.

The extension portion 113 is folded in a downward direction such that the extension portion 113 curves under the at least one back layer 30 such that a top surface of the extension part forms a secondary bottom surface of the gusset performance structure 100.

The bonding components 40a to 40c can be the same or different, and can include any suitable bonding material, including but not limited to bonding tape, adhesive, glue, or combinations thereof.

To achieve the construction as shown in FIG. 1 and as described above, the layer 50 is placed in a position above the top surface of layer 10. The bonding element 60 is then applied to connect the bottom surface of layer 50 to the top surface of extension portion 113. After the layer 50 is connected to the extension portion 113, the gusset performance structure 100 is inverted such that the layer 50 is moved to a position below the bottom surface of layer 30, the top surface of layer 50 faces the bottom surface of layer 30 and the bottom surface of layer 50 becomes the outermost surface. The extension portion 113 is folded between layer 30 and layer 50 and positioned under the bottom surface of layer 30 and above the top surface of layer 50.

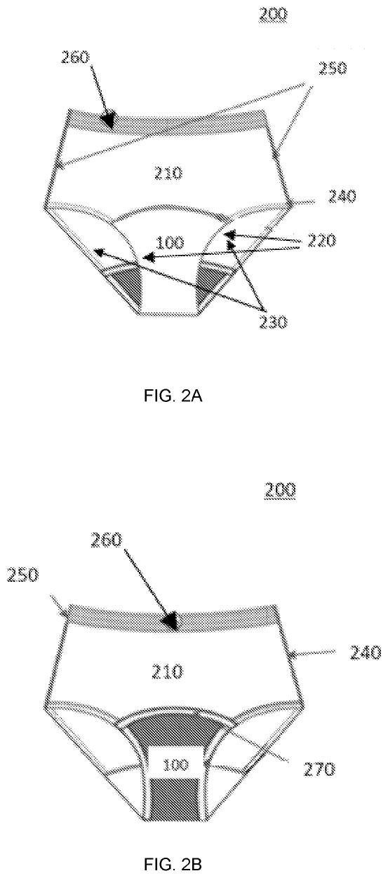

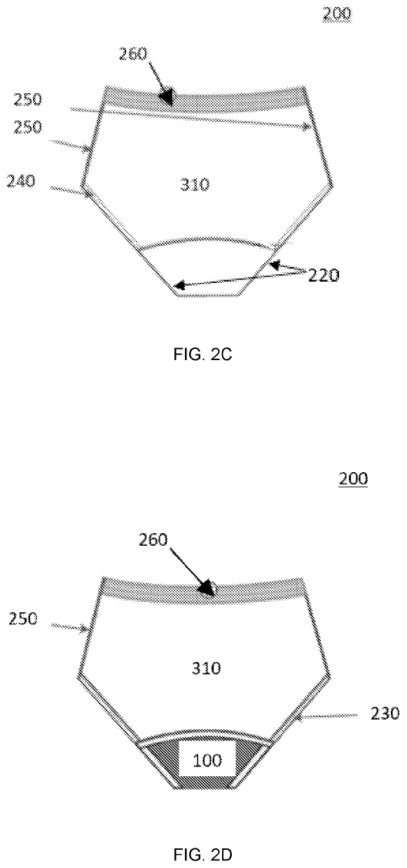

FIGS. 2A to 2D are illustrations of a garment including a gusset performance structure 100 according to an exemplary embodiment.

FIG. 2A is a front outer view of the garment 200. As illustrated in FIG. 2A, the garment 200 includes a front body portion 210 and the gusset performance structure 100, which are connected via a stitching pattern 220. The garment 200 also includes a pair of leg openings 230, each of which is surrounded by an elastic layer 240 folded into the fabric of the body portion 210 and held in place via a stitching pattern. The front body portion 210 is connected to a rear body portion 310 (illustrated in FIGS. 2C and 2D) via side seams 250. In certain embodiments, leg openings 230 do not include elastic along the edges of the gusset performance structure 100. The garment 200 can also include an elastic waistband 260.

FIG. 2B is a front inner view of the garment 200. As illustrated in FIG. 2B, the gusset performance structure 100 is connected to the front body portion 210 via a bonding element 270. The bonding element 270 can include one or more strips of fabric or can be selected from a stitching, a knitting, an adhesive layer, a bonding tape, or combinations thereof. In certain embodiments, the bonding element 270 is a stitching.

FIG. 2C is a back outer view and FIG. 2D is a back inner view of the garment 200 showing the back body portion 310 and the gusset performance structure 100. Garment features of FIGS. 2C and 2D in common with FIGS. 2A and 2B are described above and not repeated herein.

In certain embodiments, the gusset performance structure 100 is connected to the front body portion 210 and the rear body portion 310 where the bonding element 270 is a stitching. In certain other embodiments, a fabric portion can be included along sides of the gusset performance structure 100, and the fabric portion can be bonded to the gusset performance structure 100 using a bonding element 270 that is a stitching, a knitting, an adhesive layer, a bonding tape, or combinations thereof, but in this embodiment, the gusset performance structure 100 is connected to the front body portion 210 and the rear body portion 310 using a bonding element 270 that is a stitching.

The arrangement of the gusset performance structure 100 in the garment 200 is such that when the gusset performance structure 100 is inverted (turned inside-out/used in a “bag out” construction, i.e., a structure in which right sides of a structure are sewn together and then turned inside out), the stitching on the sides of the gusset performance structure 100 adjacent the leg openings 230 (e.g., the edge stitching 220) is not visible and that portion of the leg opening 230 has the appearance of being seamless.

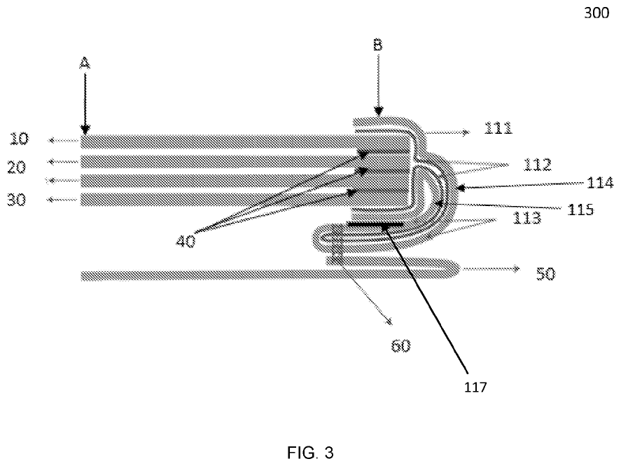

FIG. 3 is a front view of a gusset performance structure according to an exemplary embodiment of the subject application. In the embodiment shown in FIG. 3, all of the numbering of the elements is identical to the numbering in FIG. 1, with an additional bonding element 117 which is positioned between the lower part of the curved portion 111 and the second layer 115. The additional bonding element 117 is used to retain the extension portion 113 in a position beneath the lower part of the curved portion 111 and the bottom surface of the bottom layer 30. Bonding element 117 can be selected from a stitching, a knitting, an adhesive layer, a bonding tape, or combinations thereof.

The gusset performance structure according to any one of the prior exemplary embodiments can be combined with one or more material layers independently selected from fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-static®, Tencel™, lyocell, modal, or combinations thereof. However, material of any one or more of the material layers is not limited to comprising the above-described materials, and any suitable material may be incorporated into the garment.

The thickness of each of the material layers can be from about 0.2 mm to about 10 mm, preferably from about 0.3 mm to 9 mm, from about 0.4 mm to about 8 mm, from about 0.5 mm to about 7 mm, from about 0.6 mm to about 6 mm, from about 0.7 mm to about 5 mm, from about 1.0 mm to about 4.5 mm, from about 2 mm to about 4 mm, from about 3 mm to about 3.5 mm, and more preferably, about 0.1 mm, about 0.2 mm, about 0.3 mm, about 0.4 mm, about 0.5 mm, 0.6 mm, about 0.7 mm, about 0.8 mm, about 0.9 mm, about 1 mm, about 1.25 mm, about 1.5 mm, about 1.75 mm, about 2.0 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, about 5.25 mm, about 5.5 mm, about 5.75 mm, about 6 mm, about 6.25 mm, about 6.5 mm, about 6.75 mm, about 7 mm, about 7.25 mm, about 7.5 mm, about 7.75 mm, about 8 mm, about 8.25 mm, about 8.5 mm, about 8.75 mm, about 9 mm, about 9.25 mm, about 9.5 mm, about 9.75 mm, about 10 mm, or any other suitable thickness within the foregoing ranges or amounts.

The weight of the gusset performance structure can be about 50 g to about 500 g, about 100 g to about 400 g, about 200 g to about 300 g, about 210 g to about 290 g, about 220 g to about 280 g, about 230 g to about 270 g, about 230 g to about 260 g, about 240 g to about 250 g, and any other suitable weight within these ranges. For example, the weight of the gusset performance structure can be about 50 g, about 60 g, about 70 g, about 80 g, about 90 g, about 100 g, about 110 g, about 120 g, about 130 g, about 140 g, about 150 g, about 160 g, about 170 g, about 180 g, about 190 g, about 200 g, about 210 g, about 220 g, about 230 g, about 240 g, about 250 g, about 260 g, about 270 g, about 280 g, about 290 g, about 300 g, about 310 g, about 320 g, about 330 g, about 340 g. about 350 g, about 360 g, about 370 g, about 380 g, about 390 g, about 400 g, about 410 g, about 420 g, about 430 g, about 440 g, about 450 g, about 460 g, about 470 g, about 480 g, about 490 g, about 500 g, or any other weight in between the foregoing amounts or ranges.

The narrowest portion of the gusset performance structure has a width of about 0.5 inches to about 6 inches, about 2.0 inches to about 5.0 inches, preferably from about 2.5 inches to about 4.5 inches, about 3.0 inches to about 4.0 inches, about 3.2 inches to about 3.8 inches, and any other suitable widths within these ranges. For example, the width of the gusset performance structure can be about 0.5 inches, about 0.6 inches, about 0.7 inches, about 0.8 inches, about 0.9 inches, about 1.0 inch, about 1.1 inches, about 1.2 inches, about 1.3 inches, about 1.4 inches, about 1.5 inches, about 1.6 inches, about 1.7 inches, about 1.8 inches, about 1.9 inches, about 2.0 inches, about 2.1 inches, about 2.2 inches, about 2.3 inches, about 2.4 inches, about 2.5 inches, about 2.6 inches, about 2.7 inches, about 2.8 inches, about 2.9 inches, about 3.0 inches, about 3.1 inches, about 3.2 inches, about 3.3 inches, about 3.4 inches, about 3.5 inches, about 3.6 inches, about 3.7 inches, about 3.8 inches, about 3.9 inches, about 4.0 inches, about 4.1 inches, about 4.2 inches, about 4.3 inches, about 4.4 inches, about 4.5 inches, about 4.6 inches, about 4.7 inches, about 4.8 inches, about 4.9 inches, about 5.0 inches, about 5.1 inches, about 5.2 inches, about 5.3 inches, about 5.4 inches, about 5.5 inches, about 6 inches, or any other width in between the foregoing amounts or ranges.

The widest portion of the gusset performance structure has a width of about 3.0 inches to about 14.0 inches, about 4.0 inches to about 12.0 inches, preferably from about 4.5 inches to about 11.5 inches, about 5.0 inches to about 11.0 inches, about 5.5 inches to about 10.5 inches, about 6 inches to about 10 inches, and any other suitable widths within these ranges. For example, the width of the gusset performance structure can be about 3.0 inches, about 3.1 inches, about 3.2 inches, about 3.4 inches, about 3.5 inches, about 3.6 inches, about 3.7 inches, about 3.8 inches, about 3.9 inches, about 4.0 inches, about 4.2 inches, about 4.4 inches, about 4.6 inches, about 4.8 inches, about 5.0 inches, about 5.2 inches, about 5.4 inches, about 5.6 inches, about 5.8 inches, about 6.0 inches, about 6.2 inches, about 6.4 inches, about 6.6 inches, about 6.8 inches, about 7.0 inches, about 7.2 inches, about 7.4 inches, about 7.6 inches, about 7.8 inches, about 8.0 inches, about 8.2 inches, about 8.4 inches, about 8.6 inches, about 8.8 inches, about 9.0 inches, about 9.2 inches, about 9.4 inches, about 9.6 inches, about 9.8 inches, about 10.0 inches, about 10.2 inches, about 10.4 inches, about 10.6 inches, about 10.8 inches, about 11.0 inches, about 11.2 inches, about 11.4 inches, about 11.6 inches, about 11.8 inches, about 12.0 inches, about 12.2 inches, about 12.4 inches, about 12.6 inches, about 12.8 inches, about 13 inches, about 13.2 inches, about 13.4 inches, about 13.6 inches, about 13.8 inches, about 14 inches, or any other width in between the foregoing amounts or ranges.

The gusset performance structure according to any one of the prior exemplary embodiments can be incorporated into any such material layer.

The gusset performance structure according to any one of the prior exemplary embodiments can be bonded or adhered to any such material layer via a stitching, a knitting, an adhesive layer or a bonding tape.

The moisture-wicking material according to any one of the prior exemplary embodiments is selected from fabric, natural yarn, synthetic yarn, synthetic fabric, polyurethane, wool, polypropylene, polyamide, polyester or combinations thereof. The moisture-wicking material may be any material that allows one-way transport of moisture, water and/or liquids. A layer comprising the moisture-wicking material may be a water/moisture absorbing layer, such as a polymer blend, including but not limited to a polyester/nylon blend (e.g., 80% polyester, 20% nylon). In various exemplary embodiments, the moisture absorbing layer may include a combination of materials such as, but not limited to, 100% polyester, 96% cotton/4% spandex, 80% polyester/20% nylon, Bramante®/cotton blend, cotton flock, natural fibers, wool, and the like. In various exemplary embodiments, the moisture-wicking material may be a combination of materials selected from fabric, natural yarn, synthetic yarn, synthetic fabric, polyurethane, wool, polypropylene, polyamide, polyester or combinations thereof. Various, non-limiting, exemplary embodiments of the moisture-wicking layer are listed in

TABLE 1

| Composition | Weight (g) | Thickness (mm) |
|---|---|---|
| 88 Polyester/12 Lyocell | 175 | 120 |
| 45 Polyester/8 Elastane/47 Cotton | 305 | 165 |
| 80 Polyester/20 Elastane | 220 | 140 |
| 27 Cotton/73 Polyester | 140 | 160 |
| 68 Supima Cotton/26 Polyester/6 | 170 | 165 |
| Elastane | | |
| 71 Modal/21 Polyester/8 Elastane | 160 | 135 |
| 68 Lyocell/25 Polyester/7 Elastane | 170 | 170 |
| 90 Polyester/10 Elastane | 195 | 125 |
| 96 Micro Modal Air Jet/4 Elastane | 160 | 155 |
| 48 Micromodal Compact/41 Polyamide/ | 180 | 151 |
| 11 Elastane | | |
| 34 Supima Cotton/34 Micromodal Air/ | 205 | 130 |
| 32 Elastane | | |
| 43 Viscose/39 Micromodal Compact/ | 200 | 150 |
| 18 Elastane | | |
| 39 Cotton/22 Seacell/34 Modal/5 | 180 | 163 |
| Spandex | | |
| 82.7 Seacell Modal/12.3 Polyester/5 | 160 | 164 |
| Spandex | | |
| 95Ecovero Viscose/5 Spandex | 150 | 160 |
| 56 Modal/37 Refibra/7 Spandex | 170 | 160 |
| 89 Repreve Recycled Polyester/11 | 150 | 147 |
| Recycled Spandex | | |

However, the moisture-wicking material is not limited thereto, and any suitable moisture-wicking material may be incorporated into the garment. For example, the material may include a polyester, a polyamide, a viscose, a cotton, a polyester/lyocell blend, a polyester/elastane blend, a polyester/cotton blend, a polyester/cotton/elastane blend, a polyester/modal/elastane blend, a polyester/lyocell/elastane blend, a modal/elastane blend, a viscose/micromodal/elastane blend, micromodal/polyamide/elastane blend, a cotton/micromodal/elastane blend, a cotton/modal/seacell/elastane blend, a seacell modal/polyester/spandex blend, a viscose/elastane blend, a modal/refibra/elastane blend, recycled polyester/recycled elastane blend, polyamide/elastane blend, or any combinations of the foregoing.

The moisture wicking material, according to any one of the prior exemplary embodiments, can be combined with one or more material layers independently selected from fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-static®, Tencel™, lyocell, modal, or combinations thereof. However, material of any one or more of the material layers is not limited to comprising the above-described materials, and any suitable material may be incorporated into the moisture wicking material.

In certain embodiments, the moisture wicking material and/or the garment includes a moisture wicking finish. In certain embodiments, the moisture wicking material, any of the other layers and/or the garment includes an antimicrobial treatment, an antimicrobial coating, or an antimicrobial finish. In certain embodiments, the moisture wicking material, any of the other layers, and/or the garment includes an odor control treatment, an odor control coating, or an odor control finish.

The device according to any one of the prior exemplary embodiments, wherein a volume capacity of the one or more layers comprising the moisture-wicking material or waterproof material is in a range of from about 5 ml to about 550 ml, from about 55 ml to about 500 ml, from about 60 ml to about 450 ml, from about 65 ml to about 400 ml. For example, the volume capacity can be about 5 ml, about 10 ml, about 15 ml, about 20 ml, about 25 ml, about 30 ml, about 35 ml, about 40 ml, about 45 ml, about 50 ml, about 55 ml, about 60 ml, about 65 ml, about 70 ml, about 75 ml, about 80 ml, about 85 ml, about 90 ml, about 95 ml, about 100 ml, about 105 ml, about 110 ml, about 115 ml, about 120 ml, about 125 ml, about 130 ml, about 135 ml, about 140 ml, about 145 ml, about 150 ml, about 155 ml, about 160 ml, about 165 ml, about 170 ml, about 175 ml, about 180 ml, about 185 ml, about 190 ml, about 195 ml, about 200 ml, about 205 ml, about 210 ml, about 215 ml, about 220 ml, about 225 ml, about 230 ml, about 235 ml, about 240 ml, about 245 ml, about 250 ml, about 255 ml, about 260 ml, about 265 ml, about 270 ml, about 275 ml, about 280 ml, about 285 ml, about 290 ml, about 295 ml, about 300 ml, about 305 ml, about 310 ml, about 315 ml, about 320 ml, about 325 ml, about 330 ml, about 335 ml, about 340 ml, about 345 ml, about 350 ml, about 355 ml, about 360 ml, about 365 ml, about 370 ml, about 375 ml, about 380 ml, about 385 ml, about 390 ml, about 395 ml, about 400 ml, about 405 ml, about 410 ml, about 415 ml, about 420 ml, about 425 ml, about 430 ml, about 435 ml, about 440 ml, about 445 ml, about 450 ml, about 455 ml, about 460 ml, about 465 ml, about 470 ml, about 475 ml, about 480 ml, about 485 ml, about 490 ml, about 495 ml, about 500 ml, about 505 ml, about 510 ml, about 515 ml, about 520 ml, about 525 ml, about 530 ml, about 535 ml, about 540 ml, about 545 ml, about 550 ml, about 555 ml, about 560 ml, about 565 ml, about 570 ml, about 575 ml, about 580 ml, about 585 ml, about 590 ml, about 595 ml, about 600 ml, or any other suitable volume within these ranges.

The gusset performance structure according to any of the prior exemplary embodiments can be included in a garment, which includes without limitation any item that is worn, including but not limited to, an underwear, a brief, an undergarment, a bathing suit, a pajama, a shorts, a leotard, a legging, a pants, a skort, a panty, a boy short, a bikini, a thong, an overalls, a bodysuit, a leotard, a tights, a pantyhose, a boxers, a biking shorts, or a compression shorts. A material of any such garment can include, but is not limited to, fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-static®, Tencel™, lyocell, modal, or combinations thereof.

In some embodiments, various functions or acts can take place at a given location and/or in connection with the operation of one or more apparatuses or systems. In some embodiments, a portion of a given function or act can be performed at a first device or location, and a remainder of the function or act can be performed at one or more additional devices or locations.

The corresponding structures, materials, acts, and equivalents of all means or step plus function elements in the claims below are intended to include any structure, material, or act for performing the function in combination with other claimed elements as specifically claimed. The embodiments were chosen and described in order to best explain the principles of the disclosure and the practical application, and to enable others of ordinary skill in the art to understand the disclosure for various embodiments with various modifications as are suited to the particular use contemplated.

The diagrams, images and drawings depicted herein are illustrative. There can be many variations to the diagram or the steps (or operations) described therein without departing from the spirit of the disclosure. For instance, the steps can be performed in a differing order or steps can be added, deleted or modified. All of these variations are considered a part of the disclosure. It will be understood that those skilled in the art, both now and in the future, can make various improvements and enhancements which fall within the scope of the claims which follow.

The description of this disclosure has been presented for purposes of illustration and description, but is not intended to be fully exhaustive and/or limited to the disclosure in the form disclosed. Many modifications and variations in techniques and structures will be apparent to those of ordinary skill in an art without departing from a scope and spirit of this disclosure as set forth in the claims that follow. Accordingly, such modifications and variations are contemplated as being a part of this disclosure. A scope of this disclosure is defined by various claims, which include known equivalents and unforeseeable equivalents at a time of filing of this disclosure.

1. An article comprising:
a first part; and
a second part,
wherein the first part comprises:
at least one top layer having a first edge portion;
at least one intermediate layer having a second edge portion; and
at least one back layer having a third edge portion, and
wherein the second part comprises:
a first curved portion;
an extension portion;
a second curved portion;
an elastic portion; and
a garment layer,
wherein the extension portion extends from the first curved portion and from the second curved portion,
wherein the first curved portion overlaps a top surface of at least the first edge portion of the at least one top layer and the second curved portion overlaps a bottom surface of at least the third edge portion of the at least one back layer, and
wherein the elastic portion overlaps and is coupled to the extension portion and the garment layer.

2. The article of claim 1, wherein the first edge portion of the at least one top layer and the third edge portion of the at least one back layer and the second edge portion of the at least one intermediate layer are connected via a first bonding component.

3. The article of claim 1, wherein the first curved portion of the second part is connected to the at least one top layer via a second bonding component.

4. The article of claim 1, wherein the second curved portion of the second part is connected to at least one back layer via a third bonding component.

5. The article of claim 1, wherein the at least one top layer is bonded to the at least one intermediate layer via a fourth bonding component.

6. The article of claim 1, wherein the at least one at least one back layer is bonded to the at least one intermediate layer via a fifth bonding component.

7. The article of claim 1, wherein the extension part comprises two layers that are connected via a sixth bonding component.

8. The article of claim 7, wherein the first to sixth bonding components are the same or different, and are independently selected from bonding tape, adhesive, glue or combinations thereof.

9. The article of claim 1, wherein the garment layer comprises an edge portion, and wherein the edge portion of the garment layer is coupled to the extension portion and the elastic portion via a stitching pattern.

10. The article of claim 1, wherein the at least one top layer is a wicking layer.

11. The article of claim 10, wherein a material of the wicking layer is selected from fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-Static®, Tencel™, lyocell, modal, or combinations thereof.

12. The article of claim 1, wherein the at least one back layer is a waterproof layer.

13. The article of claim 12, wherein a material of the waterproof layer is selected from a water resistant, a water repellant material or a combination thereof.

14. The article of claim 1, wherein a material of the at least one intermediate layer is selected from fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-static®, Tencel™, lyocell, modal, or combinations thereof.

15. The article of claim 7, wherein a material of the garment layer is selected from fabric, natural yarn, synthetic yarn, synthetic fabric, polyurethane, cotton, silk, polyester, polyamide, elastane, rubber, plastic, metal, wool, merino wool, polypropylene, rayon, linen, bamboo, Gore-Tex®, X-Static®, Tencel™, lyocell, modal, or combinations thereof.

16. The article of claim 1, wherein a weight of the article is about 50 g to about 500 g.

17. The article of claim 1, wherein a width of the article is about 0.5 inches to about 6.0 inches at a narrowest point of the article and about 3.0 inches to about 14.0 inches at a widest point of the article.

18. The article of claim 1, wherein a water absorption capacity of the article is about 250 ml to about 1000 ml.

19. A garment, comprising the article of claim 1.

20. A device comprising:
a garment, wherein the garment comprises:
a crotch area including the article of claim 1,
a front body portion,
a rear body portion, and
a pair of leg openings.

21. The device according to claim 20, further comprising an elastic waistband.

22. The device according to claim 1, wherein each of the pair of leg openings other than the curved portion and the extension portion is surrounded by the elastic portion folded onto the body portion and connected to the body portion via a leg opening bonding component, a stitching or a bonding component and a stitching.

23. The device according to claim 1, wherein the front body portion is connected to the crotch area via a front bonding component.

24. The device according to claim 1, wherein the rear body portion is connected to the crotch area via a rear bonding component.

25. The device according to claim 1, wherein the front bonding component and the rear bonding component and the leg opening component are the same or different and are selecting from a stitching, a knitting, an adhesive layer, a bonding tape, or combinations thereof.

26. A method comprising:
causing a garment to include a crotch area;
causing the crotch area to comprise: a first part; and a second part;
causing the first part to comprise: at least one top layer having a first edge portion; at least one intermediate layer having a second edge portion; and at least one back layer having a third edge portion;
causing the second part to comprise: a first curved portion; an extension portion; a second curved portion; an elastic portion; and a garment layer;
causing the extension portion to extend from the first curved portion and from the second curved portion;
causing the first curved portion to overlap a top surface of at least the first edge portion of the at least one top layer and the second curved portion to overlap a bottom surface of at least the third edge portion of the at least one back layer; and
causing the elastic portion to overlap and be coupled to the extension portion and the garment layer.
